(19) United States
(12) Patent Application Publication
(10) Pub. No.: US 2005/0010239 A1
(43) Pub. Date: Jan. 13, 2005

(54) Hernia mesh-device with tissue adhesive

(76) Inventor: Allen B. Chefitz, New Rochelle, NY (US)

Correspondence Address: Allen B. Chefitz, M.D., Suite 301, 140 Lockwood Avenue, New Rochelle, NY, 10801, US

(21) Appl. No.: 10/784,532

(22) Filed: Feb. 23, 2004

Related U.S. Application Data
(60) Provisional application No. 60/448,726, filed on Feb. 21, 2003.

(51) Int. Cl. A61B 17/08; A61D 1/00

(52) U.S. Cl. 606/151

(57) ABSTRACT

A mesh-device for surgical repair of a hernia which has embedded within it a tissue adhesive. The tissue adhesive is activated after the mesh-device has been placed in the patient's body, and it prevents the mesh-device from changing the position of its placement without the use of sutures, staples, or other securing apparatus.

### CROSS REFERENCE TO RELATED APPLICATION

This application claims priority to U.S. Provisional Application Ser. No. 60/448,726, which was filed on Feb. 21, 2003.

### BACKGROUND OF THE INVENTION

There are many types of mesh patches or prosthetic devices used to repair hernia defects. Examples of mesh-devices include, but are not limited to, polypropylene mesh (Marlex, Bard's Perfix Plug & Patch, Ethicon's PHS, USSC's Surgipro), PTFE, Alloderm (Lifecell), and Surgisis (Cook). To prevent patches from shifting, migrating, rolling-up its edges, folding, or changing the position of its placement, one usually secures the patch in place with sutures, tacks, or staples. Those methods of securing the patch can cause pain and nerve entrapment. This invention secures the mesh in place with a tissue adhesive embedded within the prosthetic device. Examples of tissue adhesives include, but are not limited to, fibrin glues, Tisseel, Floseal, Bioglue, Thorex, polyethylene glycol, and bovine or human derived thrombin, fibrinogen, and collagen.

### SUMMARY OF THE INVENTION

This invention involves a mesh-device used to surgically repair a hernia. In particular, this mesh contains a tissue adhesive incorporated into the mesh, thereby eliminating the need for sutures, staples or any other securing apparatus.

### BRIEF DESCRIPTION OF THE DRAWING

FIG. 1 shows a mesh-device according to the present invention.

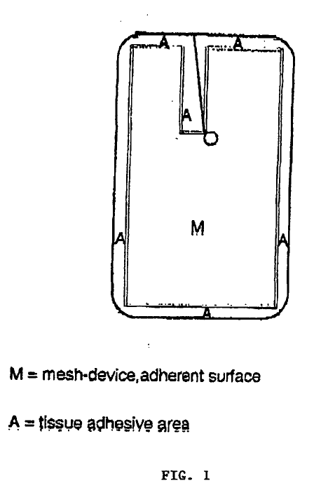

### DESCRIPTION OF THE INVENTION

The hernia mesh-device contains a tissue adhesive on its surface that is to be most adherent to the body tissue. Once placed into the patient's body space, the tissue adhesive within the mesh is activated, thereby securing the mesh in place.

1. A mesh-device used to surgically repair a hernia, comprising a tissue adhesive and a mesh patch or prosthetic device.
